(19) United States
(12) Patent Application Publication
(10) Pub. No.: US 2026/0051408 A1
(43) Pub. Date: Feb. 19, 2026

(54) SYSTEM

(71) Applicant: SOFTBANK GROUP CORP., Tokyo (JP)

(72) Inventor: Shinsuke HOSOI, Tokyo (JP)

(73) Assignee: SOFTBANK GROUP CORP., Tokyo (JP)

(21) Appl. No.: 19/299,465

(22) Filed: Aug. 14, 2025

(30) Foreign Application Priority Data
Aug. 16, 2024 (JP) 2024-137156

(51) Int. Cl. G16H 50/30; G06Q 50/02

(52) U.S. Cl. CPC G16H 50/30; G06Q 50/02

(57) ABSTRACT

A system includes a processor that receives data collected from sensors installed in a livestock farm, preprocesses the received sensor data and converts it into a format suitable for analysis, analyzes, using artificial intelligence, abnormal behavior and body temperature changes based on the preprocessed data and calculates a risk score, generates and notifies an alert when the risk score exceeds a certain threshold, proposes preventive actions and countermeasures based on the risk score, and collects feedback data from a user and updates an artificial intelligence model based on the feedback data.

### CROSS-REFERENCE TO RELATED APPLICATION

This application is based on and claims priority under 35 USC 119 from Japanese Patent Application No. 2024-137156 filed on Aug. 16, 2024, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

In conventional poultry farming environments, the early detection and prevention of avian influenza outbreaks is extremely challenging due to the lack of real-time monitoring systems and the significant difficulty in identifying infectious symptoms at an early stage. Delays in detection and intervention commonly lead to widespread infections, resulting in severe economic losses and public health risks. Additionally, traditional methods do not adapt or improve in accuracy based on accumulated feedback and changing farm conditions.

### SUMMARY

To address these problems, the present invention provides a system comprising a processor that receives sensor data collected from a livestock farm, preprocesses and analyzes the data using artificial intelligence to detect abnormal behavior and body temperature changes, and calculates a corresponding risk score. When the risk score exceeds a set threshold, the processor generates an alert and proposes specific preventive measures to the farm manager. Furthermore, the processor collects feedback data from the user's response to the alert and updates the artificial intelligence model accordingly, thereby continually improving the system's detection accuracy. The processor may also aggregate and analyze data from multiple farms to predict and manage overall risk situations.

“Processor” means that a hardware and/or software processing unit capable of executing instructions to perform data collection, preprocessing, analysis, alert generation, proposal of preventive actions, reception of feedback, and model updating.

“Sensor” means that a device installed in a livestock farm capable of measuring physical parameters such as temperature, humidity, or animal motion and transmitting the relevant data.

“Livestock farm” means that a facility where animals, such as poultry, are bred or raised for agricultural purposes.

“Sensor data” means that electronic information measured and output by one or more sensors regarding environmental or animal conditions at the livestock farm.

“Preprocessing” means that a series of operations to clean, filter, and convert raw sensor data into a suitable form for analysis.

“Artificial intelligence” means that a machine learning or data analysis technology used to recognize patterns, detect anomalies, evaluate risk, and adapt through feedback.

“Abnormal behavior” means that patterns of movement or activity identified by artificial intelligence as deviating from a reference or healthy state for livestock.

“Body temperature changes” means that variations in the measured body temperature of livestock which may indicate health status or potential disease.

“Risk score” means that a numerical or categorical value calculated by the system to represent the probability or severity of disease or abnormality in livestock.

“Threshold” means that a predetermined reference value for a risk score, above which specific actions—such as alerts or interventions—are triggered.

“Alert” means that a notification generated and transmitted by the system to inform the user or manager of the detection of abnormal conditions.

“Preventive actions and countermeasures” means that recommended or automated procedures intended to mitigate, isolate, or respond to detected abnormalities, such as isolation of animals or administration of medication.

“Feedback data” means that information input by the user regarding the result or status of interventions taken in response to alerts issued by the system.

“Artificial intelligence model” means that a trained computational framework which is used for analyzing data, detecting abnormalities, calculating risk scores, and which can be updated or improved based on new data or feedback.

“Aggregating data” means that the process of collecting and combining sensor data from multiple livestock farms to support broader analysis and prediction.

“Overall situation” means that the collective status, trend, or risk level derived from the aggregation and analysis of data across multiple livestock farms.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

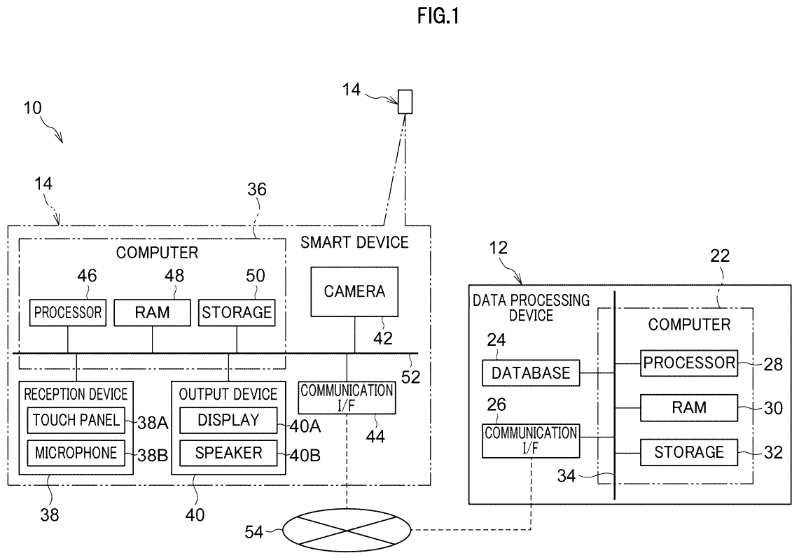

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

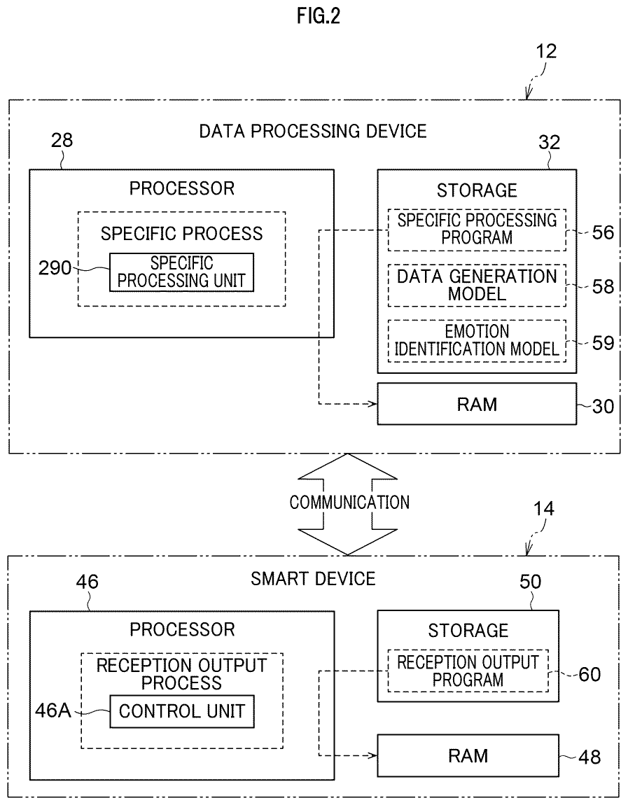

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein.

The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

In conventional observation environments, such as livestock farms or other facilities for breeding useful organisms, early detection of infectious diseases and effective prevention measures rely heavily on manual observation and incomplete data collection. This results in delays and inaccuracies in identifying abnormal behaviors or biometric changes, increasing the risk of disease outbreaks. Furthermore, responses upon abnormality detection are often inconsistent, and feedback from such interventions is rarely utilized to improve future detection or prevention systems. Additionally, there is a lack of integration and aggregation of data from multiple observation sites, resulting in missed opportunities for overall trend analysis and accurate forecasting.

The specific processing by the specific processing unit 290 of the data processing device 12 in Example 1 is realized by the following means.

The present invention provides a server comprising a processor configured to receive information from information acquisition devices installed in an observation environment, preprocess the information including normalization and removal of abnormal values, analyze the preprocessed information using a learning-enabled information analysis module to detect behavioral anomalies and biometric variations, generate and notify users with notification information and countermeasures when an evaluation index exceeds a threshold, collect feedback from users regarding executed countermeasures, update the learning-enabled module based on the feedback, manage storage of information and processing records, and facilitate information transmission between devices over a communication network. This enables real-time, accurate detection of abnormal events, automated notification with tailored recommendations, and continuous improvement of analysis accuracy through user feedback, as well as central aggregate analysis and future prediction across multiple observation environments.

The term “processor” refers to a data processing apparatus or computational unit that executes programmed instructions to perform data processing, analysis, and control tasks within the system.

The term “information acquisition device” refers to a generic data collection device, such as a sensor or camera, that is installed in an observation environment to measure or record physical or biological parameters.

The term “observation environment” refers to a controlled or monitored area, such as a livestock facility or aquaculture site, where organisms or the environment are continuously observed for data collection.

The term “preprocessing” refers to data operations including normalization, filtering, and removal of abnormal or erroneous values, conducted on raw data to prepare it for subsequent analysis.

The term “learning-enabled information analysis module” refers to a software or hardware component incorporating machine learning or artificial intelligence algorithms, which is capable of analyzing input data and improving its performance based on feedback or training data.

The term “behavioral anomaly” refers to a deviation from normal or expected behavior patterns of observed subjects, as determined by data analysis.

The term “biometric variation” refers to a change or abnormality in physiological parameters, such as body temperature or vital signs, of the observed subjects.

The term “evaluation index” refers to a numerical or categorical value calculated from analysis results, indicating the degree of abnormality or risk detected in the observation environment.

The term “notification information” refers to a message or alert generated by the system to inform a user of the detection of an abnormal event or status.

The term “user” refers to an individual, such as an operator or administrator, who receives notifications and interacts with the system, including providing feedback and executing countermeasures.

The term “countermeasure information” refers to recommended actions or procedures proposed by the system to address detected anomalies or risks in the observation environment.

The term “feedback information” refers to input provided by the user regarding outcomes or results after executing recommended countermeasures.

The term “information input device” refers to a data entry device, such as a terminal or user interface, used by the user to input feedback or other data into the system.

The term “information storage device” refers to a memory unit, database, or data storage medium where acquired information, processing results, and historical records are retained.

The term “communication network” refers to a wired or wireless network infrastructure that enables transmission of information among information acquisition devices, processing units, storage devices, and user interfaces.

The term “plurality of observation environments” refers to two or more separate observation sites or monitored areas from which data is collected and analyzed collectively by the system.

One embodiment for implementing the invention will now be described. The system includes a processor, a plurality of information acquisition devices, an information processing device (server), at least one information presentation device, an information input device, an information storage device, and a communication network. The server, or information processing device, may be implemented using a general-purpose computing apparatus such as a cloud server, edge server, or local computer equipped with at least one central processing unit (CPU) and memory. The information acquisition devices may include temperature sensors, humidity sensors, motion sensors, and imaging devices such as digital cameras or video cameras, and may be installed within a controlled observation environment such as a livestock facility, poultry house, aquaculture system, or greenhouse. Example hardware for sensors includes thermistors, digital humidity sensors, passive infrared (PIR) motion sensors, and general-purpose cameras. Example software for data handling and analysis includes Python, TensorFlow, PyTorch, OpenCV, and database management systems such as PostgreSQL.

The terminal, which integrates the information acquisition devices, periodically acquires physical and biological information from the observation environment. For instance, a temperature sensor may measure animal body temperature at intervals of ten minutes, while a camera may capture one-second video clips every second. These sensor readings and media files are temporarily stored in local memory on the terminal and are transmitted regularly (for example, every one minute) to the server via a communication network such as Wi-Fi or a wired network.

The server receives the information from the terminal and performs preprocessing using software such as Pandas and custom scripts. This preprocessing includes normalization to convert raw signals (such as voltage, analog readings, or digitized values) into standardized units, and abnormal value removal to filter out erroneous or outlier data, such as extremely high or low temperature readings. The server uses OpenCV and machine learning algorithms to extract feature data from video or image sources, classifying behaviors that may indicate abnormality, such as inactivity or erratic movement.

Following preprocessing, the server operates a learning-enabled information analysis module. This may be implemented as a machine learning model, such as a neural network trained with PyTorch or TensorFlow, which analyzes the incoming data to detect behavioral anomalies and biometric variations. The model outputs an evaluation index, such as a risk score, reflecting the probability or severity of abnormal events including infectious disease outbreaks.

When the evaluation index exceeds a predetermined reference value, the server generates notification information. The information presentation device, such as a personal computer, tablet, or smartphone, displays the notification or alert to the user and presents countermeasure information—advice or instructions suited to the abnormality detected. For example, if a risk of avian influenza is detected, the notification may suggest immediate quarantine of an animal and initiation of disinfection procedures.

The user takes action based on the countermeasure information and enters feedback information, such as actions performed and the results, via the information input device (e.g., a web or mobile application interface). The server collects this user feedback and automatically updates the machine learning model, thus enhancing the accuracy and reliability of subsequent anomaly detection.

The system is designed to aggregate and analyze data from a plurality of observation environments. This facilitates centralized monitoring of trends across multiple facilities and enables accurate forecasting of potential risks.

All acquired and processed information—including sensor readings, analysis results, alert history, user feedback, and model parameters—is systematically managed in the information storage device, which may comprise relational or NoSQL databases.

An example of a prompt sentence for the generative AI model is as follows:

“I want to design a poultry farm monitoring system that uses IoT sensors and cameras to detect avian influenza. Please give detailed steps for how the terminal collects and sends data, how server-side preprocessing and AI analysis are performed, how users receive alerts and submit feedback, and how user feedback is incorporated in model updates. Please specify what hardware and software can be used at each step.”

By using this system as described above, real-time, automated, and scalable abnormality detection and response can be realized in observation environments involving biological organisms. The system automatically adapts and improves through a feedback loop, ensuring its flexibility and long-term utility.

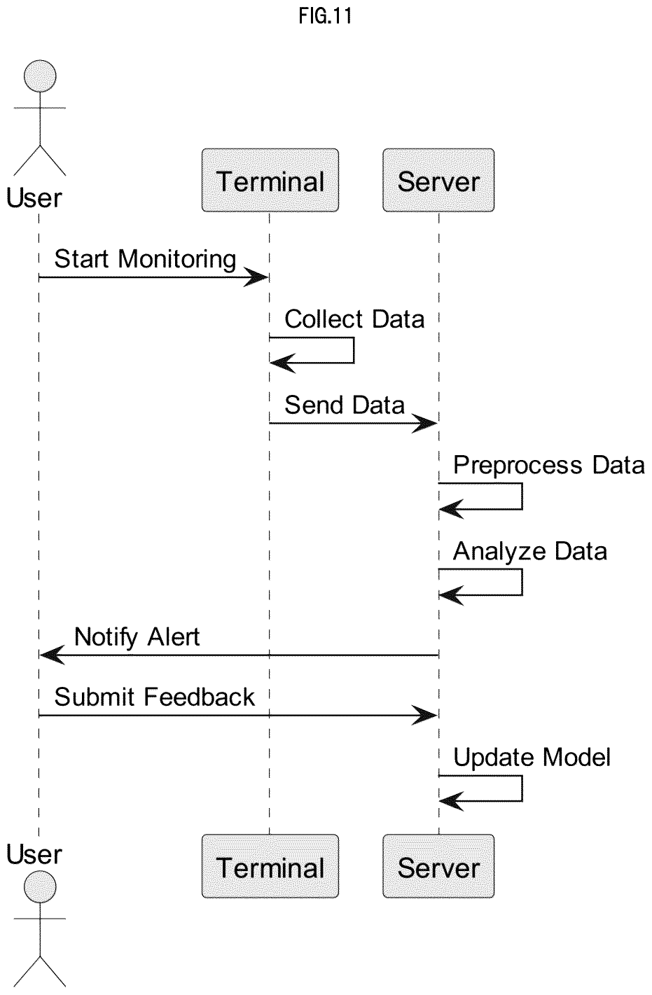

The following describes the processing flow using FIG. 11.

### Step 1:

Terminal acquires raw environmental and biological data from the observation environment using information acquisition devices such as temperature sensors, humidity sensors, motion sensors, and cameras. The input is analog or digital signals representing temperature, humidity, movement, and video frames. Terminal converts these signals into digitized records, stores them temporarily in local memory, and prepares data packets to be transmitted. The output is a set of structured data files (e.g., temperature logs, humidity readings, motion status, and video clips) ready for network transmission.

### Step 2:

Terminal transmits the collected and formatted data to the server via the communication network, for example, using Wi-Fi or wired Ethernet. The input is the data files generated in Step 1. Terminal sends these files using a secure communication protocol such as HTTPS or MQTT, and checks for successful transmission. The output is successful data delivery confirmation and transfer of data files from the terminal to the server.

### Step 3:

Server receives the transmitted data from the terminal and performs preprocessing. The input is raw data files containing sensor readings and video information. Server executes normalization (converting values to standard units), removes abnormal/outlier values that fall outside expected ranges, and splits video files into individual frames using software such as Pandas and OpenCV. The output is a set of cleaned, normalized, and labeled data suitable for subsequent analysis.

### Step 4:

Server analyzes the preprocessed data using a learning-enabled information analysis module, which may include a neural network implemented in TensorFlow or PyTorch. The input is the set of cleaned and labeled data (sensor readings and behavioral labels). Server feeds this data into the AI model, which computes features, compares them to known patterns, detects behavioral anomalies and biometric variation, and calculates an evaluation index such as a risk score. The output is a numeric or categorical risk evaluation for individual animals or groups within the observation environment.

### Step 5:

Server determines if the evaluation index exceeds a predefined threshold and, if so, generates notification information and countermeasure instructions. The input is the calculated risk score and analysis results. Server creates an alert message, detailing detected anomalies, and recommends appropriate actions based on the type of risk. The output is an alert and countermeasure message delivered to the user via the information presentation device.

### Step 6:

User receives the notification and countermeasure information through the information presentation device, such as a smartphone or computer. The input is the alert and instruction message. User reviews the recommendations, implements the suggested countermeasures (for example, isolating specific animals), and documents response actions using an information input device. The output is a feedback record containing action results and observations, ready for submission.

### Step 7:

User submits the feedback information regarding the implemented countermeasures to the server through the information input device. The input is the completed feedback form detailing actions performed and subsequent conditions. Server receives and logs this feedback information for quality improvement and history tracking. The output is successful integration of new field data into the system's feedback dataset.

### Step 8:

Server updates the learning-enabled information analysis module using the accumulated feedback data. The input is the dataset containing historical analysis results and new user feedback. Server schedules and performs retraining of the AI model, updates operational parameters, and redeploys the improved model for future analysis. The output is an updated analysis module with enhanced detection accuracy and adaptability to real-world observation environment variations.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In conventional animal breeding facilities, early detection and prevention of infectious diseases such as avian influenza rely primarily on manual monitoring or simple sensor-based data acquisition and analysis. However, these approaches often fail to provide real-time situation monitoring, timely notification to facility managers, or dynamic improvement of detection accuracy through feedback. Moreover, there is a lack of systems that can analyze complex relationships among biological and environmental data, generate risk scores using advanced artificial intelligence models, and personalize notifications or recommendations based on the manager's emotional state. As a result, outbreaks may not be detected early enough, preventive actions may be delayed, and countermeasures may not adapt to real-world situations or accumulated experience.

The specific processing by the specific processing unit 290 of the data processing device 12 in Application Example 1 is realized by the following means.

The present invention provides a server comprising a processor configured to receive information collected from measurement devices installed in multiple animal breeding facilities, preprocess and convert the received information for analysis, analyze the preprocessed data using a generative artificial intelligence model with a predetermined prompt sentence to quantify disease outbreak risk, generate alerts and notifications when risk exceeds a threshold, provide preventive or responsive actions, acquire feedback and emotional state information from the manager or user, update the generative artificial intelligence model and emotion determination engine, and send real-time advisory notifications and status reports to a management terminal. This enables dynamic, real-time monitoring, accurate and timely detection of abnormal conditions, adaptive decision support, and continuous improvement of detection precision and user engagement based on feedback and emotional context.

The term “measurement device” refers to any sensing apparatus installed in an animal breeding facility that collects biological information, environmental data, or behavioral data of the animals, including but not limited to temperature sensors, humidity sensors, motion sensors, and imaging devices.

The term “animal breeding facility” refers to any establishment, site, or structure where animals are raised, managed, or maintained for agricultural, research, or food production purposes.

The term “information” refers to any data collected by measurement devices in an animal breeding facility, including biological, environmental, and behavioral data related to the animals.

The term “preprocess” refers to any operation or computation, performed before analysis, that converts raw data into a format suitable for further analysis, including cleaning, normalization, error correction, and feature extraction.

The term “generative artificial intelligence model” refers to a processing model capable of creating outputs (such as risk assessments, responses, or analyses) based on provided input information and prompt sentences, using artificial intelligence technologies such as machine learning or deep learning frameworks.

The term “prompt sentence” refers to a structured instruction or query provided as input to the generative artificial intelligence model, which specifies the analysis objective or the task to be performed by the model.

The term “risk score” refers to a quantitative or qualitative value generated by the artificial intelligence model that represents the likelihood or degree of disease outbreak or abnormality detected among the animals.

The term “alert information” refers to any notification, message, or signal generated and transmitted by the processor when a predetermined risk threshold is exceeded, for the purpose of warning the facility manager or user about potential danger.

The term “preventive measures or responsive actions” refers to any recommended activity, guideline, or protocol provided to the facility manager or user for the purpose of preventing, mitigating, or counteracting a detected risk or abnormality.

The term “result information” refers to feedback data provided by the manager or user describing the actions taken, the outcome observed, and optionally any related animal status data.

The term “emotional state information” refers to data representing or reflecting the psychological or emotional condition of the manager or user, which may be collected via explicit input or estimated by analysis models.

The term “emotion determination engine” refers to a processing module or system that estimates, detects, or classifies the emotional state of the manager or user based on provided input or interaction data.

The term “real-time advisory notifications and status reports” refers to messages or information delivered by the processor to the management terminal without delay, which provide situational guidance, risk status, or operational advice pertinent to the breeding facility.

The term “management terminal” refers to an electronic communication device, such as a computer, tablet, or mobile phone, used by the facility manager or user to receive notifications, input feedback, and interact with the system.

The term “aggregate” refers to the process of collecting, combining, and synthesizing information from multiple sources or facilities for the purpose of comprehensive analysis or prediction.

The term “future prediction” refers to an estimation or projection of an event, condition, or outcome regarding disease occurrence or facility status, derived by the processor using historical and real-time data.

An embodiment of the present invention will be described in detail below.

The invented system enables real-time monitoring and risk management for infectious diseases, such as avian influenza, in animal breeding facilities. The core of the system comprises a server equipped with a processor and a management terminal operated by a user, along with measurement devices installed at the breeding facility. These components are connected via a network, allowing for automatic data collection, processing, analysis, notification, and feedback.

The terminal, which may include microcontrollers such as a generic embedded processing unit, is installed at an animal breeding facility. The terminal is equipped with measurement devices such as temperature sensors (for example, a commonly available digital sensor), humidity sensors, motion sensors, and imaging devices such as a camera module. The terminal is configured to periodically collect biological information, environmental data (e.g., temperature, humidity), and behavioral data (e.g., activity levels, movement patterns, image frames) from the animals and environment.

The terminal processes the acquired data by aggregating and formatting it, for instance, converting the output of each sensor into structured data using standard data formatting techniques. The formatted information is transmitted at regular intervals to the server via a communication protocol, such as HTTP, MQTT, or another standard network protocol.

The server receives this information and stores it in a database system, which may be implemented using general-purpose database software. The server preprocesses the received information by performing cleaning (e.g., removal of noise and outliers), normalization, error correction, and feature extraction. Tools such as general data analysis libraries (e.g., NumPy, Pandas) as well as libraries for image processing (e.g., OpenCV) and data storage solutions (e.g., MySQL) may be utilized for these operations.

To analyze risk and detect abnormalities, the server uses a generative AI model built with a machine learning or deep learning framework (such as TensorFlow or PyTorch). The server generates a prompt sentence and inputs both the preprocessed information and the prompt into the generative AI model. For instance, the following prompt sentence may be used:

“Given the temperature readings and activity levels of the chickens, determine the risk score for bird flu infection. Consider higher temperatures and inactivity as higher risk.”

The generative AI model analyzes the input data in the context of the prompt sentence and outputs a risk score—a quantitative assessment of infection risk. If the risk score exceeds a certain threshold, the server generates alert information that is transmitted as a notification to the manager's management terminal, such as a general-purpose smart device or computer. The server also provides specific preventive measures or responsive actions, drawing on past data and guidance protocols.

Additionally, the system allows the user (manager) to review the alerts and execute recommended actions, such as isolating animals or performing disinfection. The user then provides result information and, if applicable, emotional state information back to the server via the management terminal. For example, using a dedicated software application, the user may enter that isolation was performed and temperature returned to a normal range, as well as indicate feeling relieved.

The server aggregates this feedback and uses it to update the generative AI model and, if implemented, an emotion determination engine (which may use standard natural language processing models, such as BERT). This feedback-driven retraining ensures that the model becomes increasingly accurate and adaptive for future situation analysis.

A concrete example of operation is as follows. The terminal measures a temperature of 38.2° C. for an animal and detects no movement over a 20-minute period. The server receives the data, performs preprocessing (including noise removal and activity labeling), and applies the generative AI model with the above prompt sentence. The model outputs a high risk score (for example, 80). The server then notifies the user to isolate the animal and begin preventive measures. The user later reports that the animal's temperature normalized after isolation, and this outcome is used to update the AI model for improved future risk assessment.

All server-side processing may be performed using general-purpose computing hardware and widely available software libraries, ensuring broad compatibility and low implementation overhead. The invention therefore enables a robust, adaptive, and real-time disease monitoring and management solution for animal breeding facilities, maximizing the effectiveness of both automated analysis and human intervention.

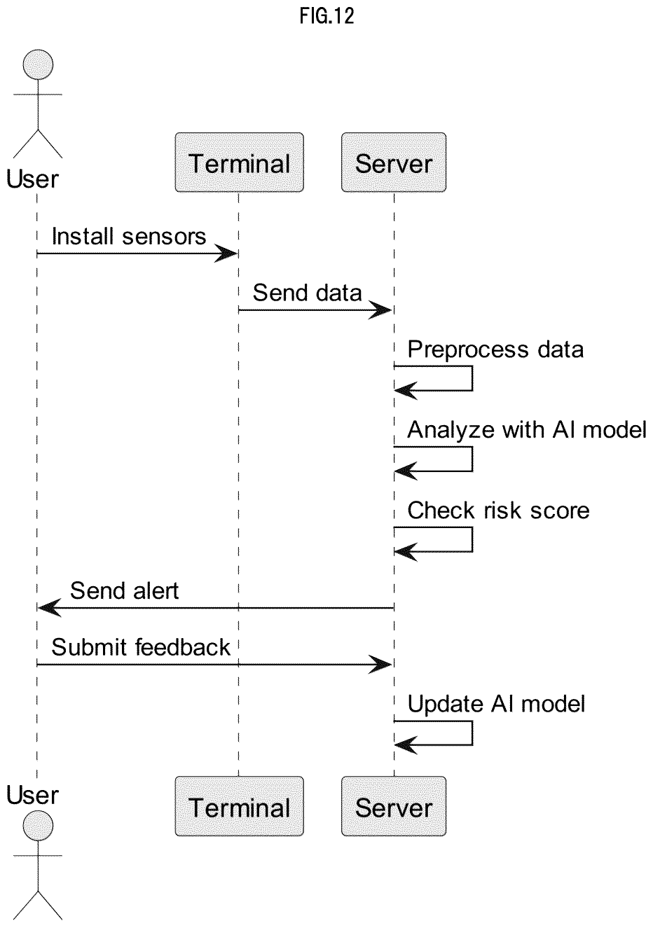

The following describes the processing flow using FIG. 12.

### Step 1:

The terminal collects biological, environmental, and behavioral data from the animal breeding facility using measurement devices such as temperature sensors, humidity sensors, motion sensors, and cameras. The input is the raw sensor signals and image files. The terminal formats these measurements into structured data (for example, creating a JSON file containing animal ID, timestamp, temperature, humidity, motion count, and image references) and stores them in a local buffer as the output.

### Step 2:

The terminal transmits the structured sensor data and image data to the server via a communication protocol such as HTTP or MQTT at predefined time intervals. The input is the collected and formatted data in the local buffer. The terminal packages the data into network packets and sends it to the server's endpoint. The output is that the server receives and stores the transmitted data.

### Step 3:

The server receives the incoming data from the terminal and stores it in a database system for further processing. The input is the transmitted sensor data and images from the terminal. The server validates the integrity of the incoming data, parses the JSON fields, assigns unique IDs, and stores each record into appropriate database tables. The output is organized, retrievable data records in the database.

### Step 4:

The server preprocesses the stored data to prepare it for analysis. The input is the raw data records in the database. The server performs data cleaning such as removal of noise and outliers from temperature and humidity values, normalization of sensor data ranges, interpolation of missing values, and feature extraction such as summarizing motion patterns or extracting animal activity indicators from image data using computer vision techniques. The output is a set of preprocessed, normalized, and annotated data records for each animal.

### Step 5:

The server uses the preprocessed data as the basis for risk analysis with a generative AI model. The input is the normalized and annotated data set and a prompt sentence such as, “Given the temperature readings and activity levels of the chickens, determine the risk score for bird flu infection. Consider higher temperatures and inactivity as higher risk.” The server encodes the input features and passes them along with the prompt to the generative AI model (e.g., running on TensorFlow or PyTorch). The model computes a risk score for each animal, reflecting the probability or severity of disease occurrence. The output is a risk score for each animal.

### Step 6:

The server checks if the risk score of any animal exceeds the predefined alert threshold. The input is the set of risk scores and the predetermined threshold value. If the score is above the threshold, the server generates alert information and a notification message, which includes specific prevention or response actions. For instance, the server may craft a message: “Abnormal behavior and high temperature detected. Isolate the animal and start quarantine procedures.” The output is an alert and countermeasure message sent to the management terminal.

### Step 7:

The server, upon generating an alert, sends notifications in real-time to the management terminal operated by the user via push notification services, email, or other communication means. The input is the alert and prevention instructions. The server invokes the corresponding messaging API, transmitting the message content to the user's device. The output is the appearance of a new notification on the user's device.

### Step 8:

The user receives the alert and prevention instructions on the management terminal and takes the recommended actions, such as isolating a specific animal or starting disinfection procedures. The input is the notification content received on the terminal. The user observes and implements the instructions in the breeding facility. The output is the new status or outcome in the animal environment.

### Step 9:

The user reports the outcome and, if applicable, their emotional state back to the server using the management terminal's feedback interface. The input is the observed result and emotional feedback, entered through the user interface. The device packages this feedback as a structured response and sends it to the server. The output is that the server receives new feedback records.

### Step 10:

The server stores the feedback information and updates the database. The input is the feedback data returned from the user, including action results and emotional state information. The server parses and validates the received information and appends it to the relevant feedback tables in the database for future analysis and learning. The output is an updated set of training and outcome data records.

### Step 11:

The server periodically retrains or fine-tunes the generative AI model and, if implemented, the emotion determination engine, using the accumulated feedback data. The input is the database of historical feedback records, outcome results, and user emotional states. The server uses data processing and machine learning tools (such as TensorFlow or PyTorch) to improve the AI model's accuracy, update parameters, and redeploy the latest model for risk prediction and personalized notification. The output is an updated generative AI model and emotion engine, ready for application in the next analysis cycle.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

In conventional biological cultivation management systems, early detection and prevention of infectious diseases such as avian influenza are insufficient, as these systems often provide uniform notifications to users without considering individual user conditions or emotional states. Furthermore, conventional systems lack mechanisms for leveraging user feedback and emotional data to improve the accuracy and adaptability of analysis models. As a result, users may experience stress, delayed or inappropriate responses, and reduced precision in disease risk detection and recommendations.

The specific processing by the specific processing unit 290 of the data processing device 12 in Example 2 is realized by the following means.

The present invention provides a server comprising a processor configured to receive data from measuring devices installed in biological cultivation facilities, preprocess such data for analysis, analyze behavioral and physiological abnormalities using trained models, generate and transmit alerts when risk thresholds are exceeded, propose preventive or countermeasure actions based on risk evaluation, collect user feedback and emotional state data, update analysis and emotion estimation models based on collected data, and adapt notification content according to the user's emotional state. This enables early detection of infectious symptoms, rapid and personalized recommendations, and continuous improvement of model accuracy through the incorporation of user feedback and emotional information, ultimately reducing user stress and enhancing disease prevention efficiency.

The term “processor” refers to a hardware component or integrated circuit that executes instructions or algorithms to perform computational and data processing tasks within a system.

The term “measuring device” refers to any instrument or sensor installed in a biological cultivation facility for the purpose of collecting physical, biological, or environmental information, including but not limited to temperature sensors, motion sensors, humidity sensors, or imaging devices.

The term “biological cultivation facility” refers to a physical location or environment where living organisms such as animals or plants are raised, cultivated, or managed, including, for example, a livestock farm, aquaculture farm, or greenhouse.

The term “preprocess” refers to the operations performed on raw data, such as normalization, filtering, or format conversion, in order to prepare the data for analysis by subsequent algorithms or models.

The term “identification model” refers to a computational or machine learning model that is trained to detect, classify, or recognize abnormal behaviors, physiological changes, or risk factors based on input data.

The term “risk evaluation value” refers to a numerical or categorical indicator calculated by the processor, which quantifies the assessed risk of disease occurrence or abnormality in the biological cultivation facility, based on the analyzed data.

The term “alert information” refers to a message, notification, or signal generated by the system to notify a user or external device when a calculated risk exceeds a predetermined threshold.

The term “communication terminal” refers to any device or user interface, such as a smartphone, tablet, or computer, that can receive messages, alerts, or information outputted by the system.

The term “preventive or countermeasure actions” refers to recommended interventions or procedures provided to the user, which are designed to prevent the occurrence or spread of diseases or abnormal conditions within the biological cultivation facility.

The term “feedback” refers to information provided by the user in response to an alert, recommendation, or outcome, including but not limited to action status updates, observations, or additional data entries.

The term “emotional state information” refers to information relating to a user's emotional or psychological condition, which may be derived from direct user input, behavioral patterns, linguistic cues, or system analysis.

The term “emotion estimation model” refers to a computational or machine learning model that infers or classifies a user's emotional state based on feedback, behavioral data, or other relevant input.

The term “aggregate basis” refers to the process of combining, integrating, or analyzing data from multiple biological cultivation facilities in order to assess wider trends, predict outbreaks, or provide a comprehensive evaluation.

The system comprises a processor, a plurality of measuring devices, and at least one communication terminal. The measuring devices, such as temperature sensors, humidity sensors, motion sensors, and imaging devices (for example, general thermometers, accelerometers, and digital cameras), are installed within a biological cultivation facility, such as a livestock farm or greenhouse.

The terminal is equipped with a hardware platform such as a microcomputer (for example, a single-board computer), and is programmed to regularly collect data from the measuring devices.

The terminal stores the data temporarily and periodically transmits it to the server via a communication network using a protocol such as HTTPS or MQTT. The terminal may also attach device identification and authentication data to each transmission in order to maintain data integrity and security.

The server, which includes a general-purpose processor and storage medium (for example, a virtual machine running on a data center or a cloud server), receives the measurement information. The server uses software tools such as Python, scikit-learn, or equivalent for processing the incoming data. First, the server preprocesses the data, performing normalization (for example, by using statistical methods or library functions), removal of abnormal values (such as filtering out outlier sensor readings), and format conversion (such as converting raw sensor values into structured records suitable for analysis).

After preprocessing, the server analyzes the data by employing machine learning models. The server may use, for example, a trained LSTM or other anomaly detection model to interpret time-series data for abnormal physiological or behavioral patterns. For image-based or motion analysis, the server could employ an object detection model such as YOLO, or an equivalent model, to assess abnormal behaviors. The results of these analyses are quantified by the server, which computes a risk evaluation value representing the risk of a disease outbreak or abnormal state.

If the risk evaluation value exceeds a threshold, the server generates alert information, including details of the detected anomaly and recommended actions, and transmits the alert to the user's communication terminal (such as a smartphone, personal computer, or tablet). The server maintains a protocol library for disease prevention and countermeasure actions, from which it fetches the appropriate actions to suggest. The alerts are sent using general notification services or push messaging platforms.

The user, upon receiving an alert and recommendation on their communication terminal, is able to review the details and take the recommended actions, such as isolating an affected organism or implementing a designated treatment. The user then reports back to the system using the terminal interface, providing feedback regarding the implemented actions and, optionally, contextual information about their emotional state. This information may be entered directly or inferred by the system from user behavior and language.

The server periodically collects the feedback and emotional state information and uses these inputs to update the identification model and emotion estimation model through supervised or semi-supervised learning. Machine learning frameworks such as PyTorch or TensorFlow may be employed for retraining the models. By using the latest feedback and emotional state information, the system improves the accuracy and adaptability of its disease risk analysis and notification personalization capabilities.

Furthermore, the server can aggregate information received from multiple biological cultivation facilities to analyze regional or overall trends and predict outbreaks on a larger scale.

### Concrete Example:

A user installs temperature, motion, and camera sensors in a poultry house. The terminal collects data every ten minutes and transmits it daily to the server. The server, executing a Python-based program, preprocesses and analyzes the data with an LSTM model for temperature series and YOLO for image analysis. Upon detecting a combination of high temperature and immobility, the server generates a risk score, sends an alert and isolation procedure to the user's smartphone, and requests feedback. The user performs the action, then confirms, “isolation completed, temperature returned to normal,” which the server uses to further train its detection models.

### Example Prompt Sentence for a Generative AI Model:

“Monitor the chickens' health status in real time using temperature, motion, and camera sensors.

When the server detects a risk pattern (e.g., elevated temperature and absence of motion), immediately generate a push notification with specific action steps and emotional support. After the user takes action and submits feedback, continuously update the AI models for improved anomaly detection and personalized alerting.” Through this embodiment, precise, efficient, and personalized detection and mitigation of infectious diseases or abnormal states in biological cultivation facilities can be realized.

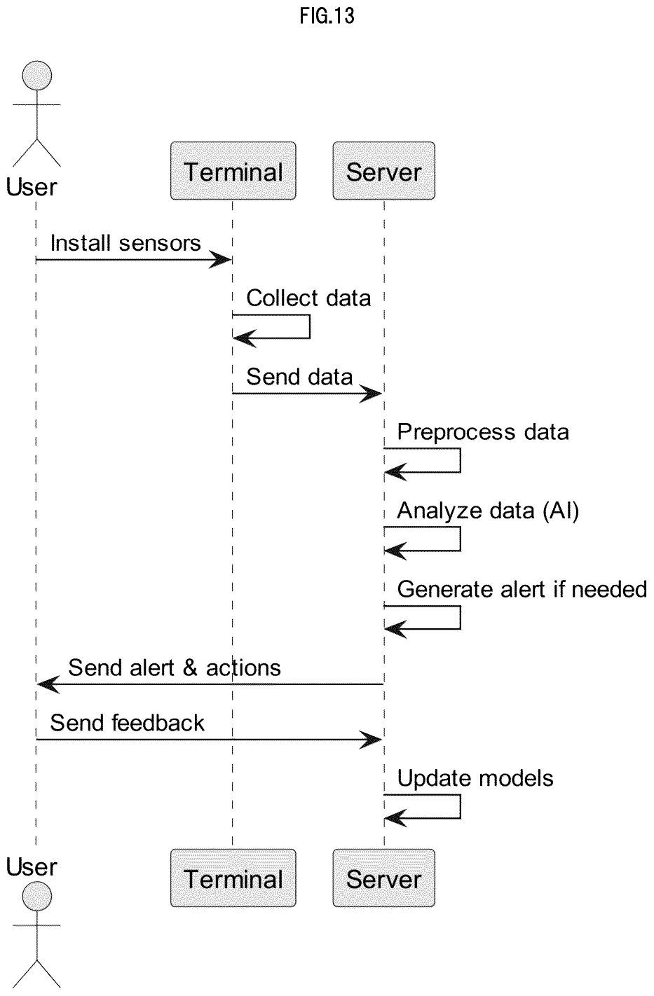

The following describes the processing flow using FIG. 13.

### Step 1:

The terminal periodically collects raw data from multiple measuring devices installed in the biological cultivation facility, such as temperature sensors, motion sensors, and imaging devices. The input is real-time sensor readings and image data. The terminal processes the input by organizing data into structured records with timestamps and device IDs, and stores them locally as files or in a buffer memory. The output is a batch of structured measurement data ready for transmission.

### Step 2:

The terminal transmits the structured measurement data to the server over a network, such as Wi-Fi or wired Ethernet, using a secure communication protocol. The input is the locally buffered batch of measurement data. The terminal formats the data as JSON or similar, attaches authentication information, and sends it via HTTP POST or equivalent. The output is successful delivery and receipt confirmation from the server.

### Step 3:

The server receives the measurement data and parses the incoming payload to extract information from temperature sensors, motion sensors, and imaging devices. The input is the transmitted batch of sensor data from the terminal. The server validates the data, confirms the source identity, and stores the parsed records in a database for processing. The output is a set of verified and registered measurement records in the server's storage.

### Step 4:

The server preprocesses the verified measurement records to prepare them for analysis. The input is the unfiltered, unnormalized measurement data stored in the database. The server performs normalization of numerical values, filters out values outside biological plausibility, and converts image data into grayscale frames if necessary. The output is a cleansed and structured dataset suitable for machine learning analysis.

### Step 5:

The server applies machine learning models, such as a time-series anomaly detection model (e.g., LSTM) for physiological data and an image analysis model (e.g., YOLO) for behavioral data, to the preprocessed dataset. The input is the cleansed measurement data and image frames. The server computes analytic results including abnormal behavior detection, unusual temperature trends, and assigns a risk evaluation score for each subject or group. The output is a set of results including detected anomalies and risk scores.

### Step 6:

The server determines if the risk evaluation score exceeds a predefined threshold value. The input is the risk scores from the AI models. If the threshold is exceeded, the server generates alert information including the nature and location of the detected anomaly. The output is an alert message and recommended actions, formatted for transmission to the user's communication terminal.

### Step 7:

The server transmits the alert message and recommended actions to the user's communication terminal via push notification or other messaging service. The input is the alert message, user contact information, and action protocol documentation. The server sends the message and protocol to the designated terminal. The output is the delivery of actionable alerts and guidance to the user.

### Step 8:

The user receives the alert and recommended actions on their communication terminal. The input is the push notification message and accompanying action protocol. The user reads the information, performs the suggested countermeasures (such as isolating a subject or applying treatment), and records the outcome and relevant observations. The output is feedback data detailing the actions taken and the observed effects.

### Step 9:

The user submits the feedback data and optionally provides information about their emotional state using the communication terminal interface. The input is the user's feedback and emotional state information. The communication terminal packages the feedback and transmits it to the server via a secure channel. The output is successful data upload to the server for analysis.

### Step 10:

The server receives, stores, and analyzes the feedback and emotional state data from the user. The input is the feedback records with emotional state annotations. The server updates the identification and emotion estimation models by retraining them with the newly collected data, using machine learning libraries such as PyTorch or TensorFlow. The output is improved and more adaptive models for subsequent processing cycles.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

In conventional biological cultivation or production facilities, early detection and prevention of abnormal events, such as equipment malfunctions or environmental abnormalities, are difficult to achieve in a timely manner. Existing systems often lack the ability to process data from multiple types of sensors, dynamically update risk evaluation methods, or customize notification contents based on the emotions or stress levels of users. Additionally, these systems may not provide sufficient support for operators in executing optimal countermeasures or integrating feedback data to improve future performance. As a result, facility operators are faced with delayed response times, insufficient preventive guidance, and decreased operational efficiency.

The specific processing by the specific processing unit 290 of the data processing device 12 in Application Example 2 is realized by the following means.

The present invention provides a server comprising a processor configured to receive measurement data from information acquisition devices installed in biological cultivation or production facilities, preprocess the data to a format suitable for analysis, analyze the data with a generative artificial intelligence model to detect abnormal states and quantify risk, generate and send alerts with recommended countermeasures to user terminals when risks are detected, receive and incorporate user response feedback including emotional states, update the artificial intelligence model accordingly, and adjust notifications based on user emotions. This enables early and accurate detection of facility abnormalities, provision of customized and effective countermeasures, and continuous improvement of the system through user feedback integration and emotional awareness.

The term “processor” refers to a hardware or software component capable of executing instructions and performing data processing operations in the system.

The term “information acquisition device” refers to any apparatus or component, such as a sensor or camera, that collects or measures physical, environmental, or operational data in a facility.

The term “biological cultivation facility” refers to any facility where living organisms, such as animals or plants, are raised, cultivated, or managed.

The term “production facility” refers to any facility or site where products are manufactured, assembled, processed, or otherwise produced.

The term “measurement data” refers to raw or processed information collected by information acquisition devices, including but not limited to temperature, humidity, vibration, or image data.

The term “preprocessing” refers to operations performed on measurement data to cleanse, normalize, classify, or otherwise prepare the data for subsequent analysis.

The term “generative artificial intelligence model” refers to any artificial intelligence algorithm or computational model, including but not limited to neural networks or machine learning models, capable of generating predictions, detecting anomalies, or modeling complex data patterns.

The term “abnormal condition” refers to any state or event within the facility that deviates from normal operational parameters and may indicate a risk or failure.

The term “index representing the degree of abnormality” refers to a numerical or categorical value computed by the system to quantify the severity or likelihood of an abnormal condition.

The term “alert” refers to a message, notification, or signal generated by the system to inform users or operators of detected abnormal conditions or risks.

The term “communication terminal” refers to any user-facing device, such as a smartphone, tablet, computer, or wearable, that can receive alerts and communicate with the system.

The term “preventive measures or countermeasures” refers to specific actions, instructions, or procedures suggested by the system to mitigate, prevent, or resolve detected abnormal conditions.

The term “user response information” refers to data submitted by the user after receiving an alert, including information on actions taken, results observed, or subjective emotional states.

The term “emotional state” refers to the psychological or emotional condition of the user, such as stress, anxiety, calmness, or confidence, as determined directly from user input or indirectly from user behavior.

The term “notification content adjustment” refers to the process of modifying the message, language, or tone of alerts and instructions provided to the user, based on their identified emotional state.

One embodiment for implementing the present invention involves a system comprising a server with an integrated processor, a network of terminals equipped with information acquisition devices, and communication terminals for user interaction.

The server can be constructed with a general-purpose computer hardware platform such as a multi-core processor (for example, a processor based on the x86 architecture) and sufficient memory and storage capacity. The operating system may include a widely used server-class platform such as Linux. The server executes software programs written in languages such as Python or Java, and utilizes standard libraries for web API handling, data processing, and machine learning. For the database, an example is a document-oriented database such as MongoDB or a relational database like PostgreSQL.

The terminals installed in the biological cultivation or production facility are fitted with various information acquisition devices. These include, but are not limited to, temperature sensors, humidity sensors, vibration sensors, and cameras for image acquisition. Each terminal may use a microcontroller or embedded processor to interface with the sensors, and have network connectivity enabled through wired Ethernet or wireless modules (such as Wi-Fi or LTE). The firmware operating on the terminals may be based on standard embedded operating systems or real-time operating systems.

The information acquisition devices periodically gather measurement data, such as temperature, humidity, vibration magnitude, and image frames. The terminal formats these data together with device identification and timestamps into data packets, which are transmitted over a network protocol (such as HTTP over TCP/IP) to the server's data intake interface.

Upon receiving measurement data, the server preprocesses the data to remove noise, normalize measurements, and classify records. For example, the server may use Pandas and NumPy for numerical data handling and OpenCV for image processing. The preprocessed data are then passed to a generative artificial intelligence model executed on the server, for example, a neural network implemented using PyTorch or TensorFlow.

The generative AI model analyzes the preprocessed sensor and image data to detect abnormal conditions, such as deviations in operational parameters or unusual patterns in image frames. The processor quantifies the degree of abnormality with an index or risk score. If this index exceeds a predetermined threshold, the server generates an alert message and sends it to communication terminals, such as smartphones (running Android or iOS), tablet computers, or wearable head-mounted displays, using push notification services.

The server also retrieves from its database a set of predefined preventive measures or countermeasures tailored to the detected abnormality, and includes this information in the notification sent to the user.

When the user receives an alert, the user operates the communication terminal to review the recommended action and implements the necessary steps at the facility. After executing the prescribed actions, the user submits feedback data through the communication terminal, describing the measures taken, the results observed, and optionally providing information regarding their emotional state or stress level.

The server receives and records this feedback, including both objective facility status and subjective emotional feedback. The feedback is used as new training data to update or retrain the generative AI model for enhanced accuracy and personal relevance in subsequent detections and notifications. The server may employ a sentiment analysis engine, such as one based on the BERT or EmotionBERT architecture, to infer the user's emotional state from textual feedback, and may adjust the tone or content of future alerts accordingly to better support the user.

A representative example of a prompt sentence for the generative AI model is as follows: Analyze the following dataset and predict the anomaly risk score for this production line. Dataset: temperature data, humidity data, vibration data, image data. Time range: 30 minutes. Output: anomaly risk score and detailed explanation of any detected abnormal events.

Through the above configuration, the embodiment provides early detection of abnormal states and timely notification to users, enables users to receive actionable guidance through their chosen devices, and continuously improves the detection algorithms through feedback loops integrating both operational outcomes and user emotions.

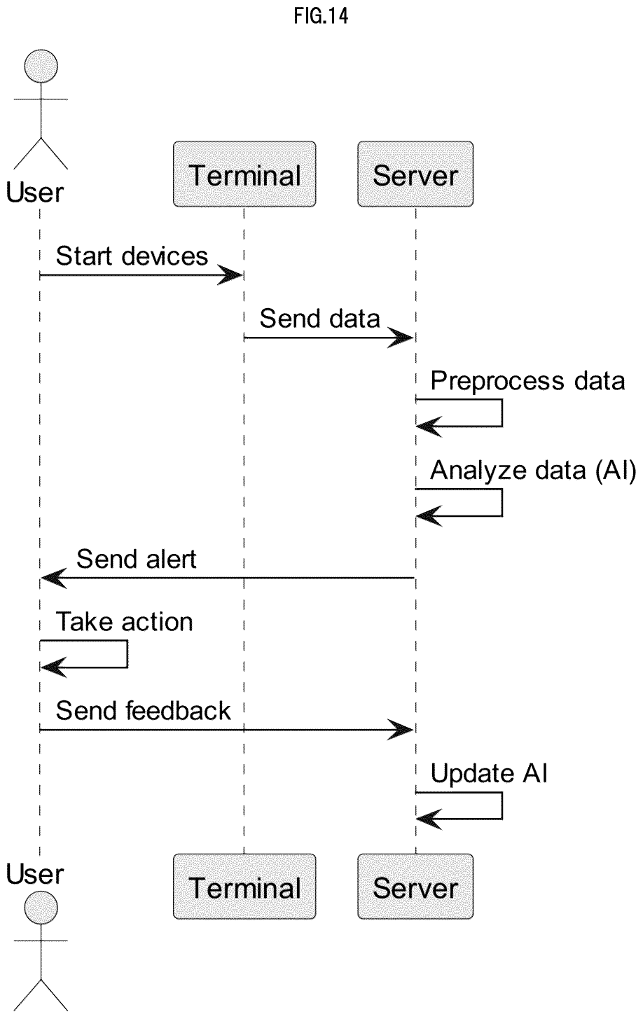

The following describes the processing flow using FIG. 14.

### Step 1:

The terminal collects raw sensor data and images from information acquisition devices such as temperature sensors, humidity sensors, vibration sensors, and cameras. The terminal formats these readings with device ID and timestamps and transmits them over the network to the server. Input: Physical environment readings. Output: Formatted sensor and image data packets sent to the server.

### Step 2:

The server receives the transmitted data from multiple terminals via its API endpoint. The server authenticates the source and verifies the data format before saving the incoming measurements and image files into a database. Input: Formatted sensor and image data packets. Output: Validated and stored raw measurement records in the database.

### Step 3:

The server performs data preprocessing on the raw measurement records by retrieving recent data from the database. The server applies noise removal (such as a moving average filter), normalizes numerical data, and processes images by resizing and label extraction. The server then creates a unified data structure prepared for analysis. Input: Raw measurement records from the database. Output: Preprocessed and aggregated data suitable for AI analysis.

### Step 4:

The server loads a generative AI model and submits the preprocessed sensor and image data as input. The model analyzes the data to detect abnormal conditions, such as unexpected temperature rises or irregular vibration patterns, and outputs a risk score that quantifies the degree of abnormality. Input: Preprocessed and aggregated data. Output: Risk score and anomaly detection results.

### Step 5:

The server evaluates whether the risk score exceeds a predefined threshold. If the threshold is exceeded, the server generates an alert containing the detected abnormality and recommended countermeasures. The alert is formatted for delivery and sent to the user's communication terminal using a suitable notification method, such as push notification or SMS. Input: Risk score and anomaly detection results. Output: Alert message and recommended actions delivered to user devices.

### Step 6:

The user receives the alert on their communication terminal, reviews the recommended actions, and performs the specified steps at the facility. The user then enters feedback, reporting the actions taken, observed outcomes, and optionally providing information about their emotional state or level of stress using an application interface. Input: Alert message and recommended actions. Output: User feedback data including action results and emotional state.

### Step 7:

The server receives the user feedback through its API and stores the data in the database. The server conducts sentiment analysis on any free-text input to infer the emotional state of the user. It then updates or fine-tunes the generative AI model using the action results and emotional state to improve future detection and notifications. The server also adjusts the language or tone of future alerts according to the inferred user emotions. Input: User feedback data and emotional indicators. Output: Updated AI model and optimized, personalized notification content for the user.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

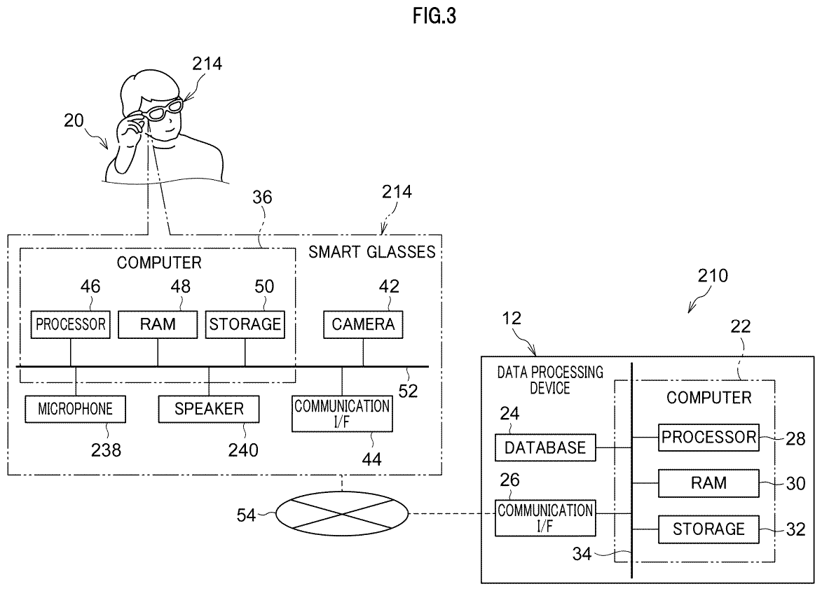

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

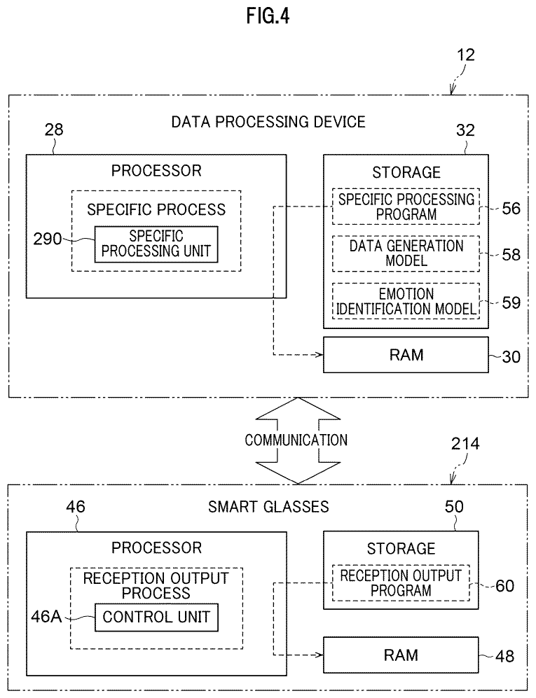

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

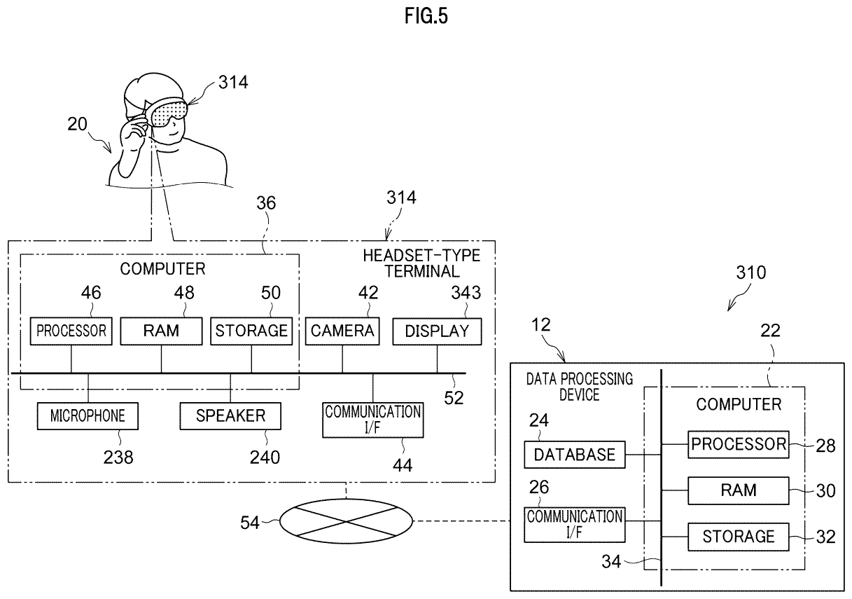

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

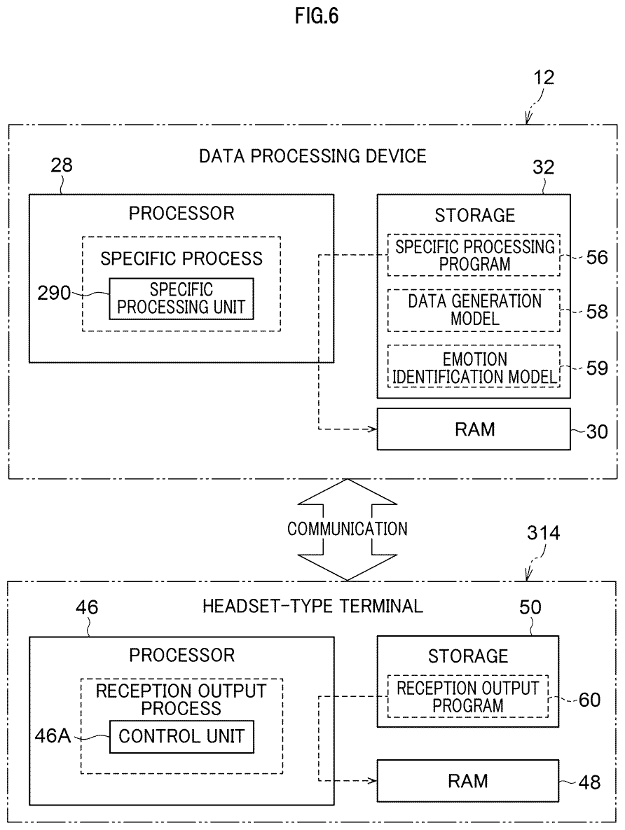

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

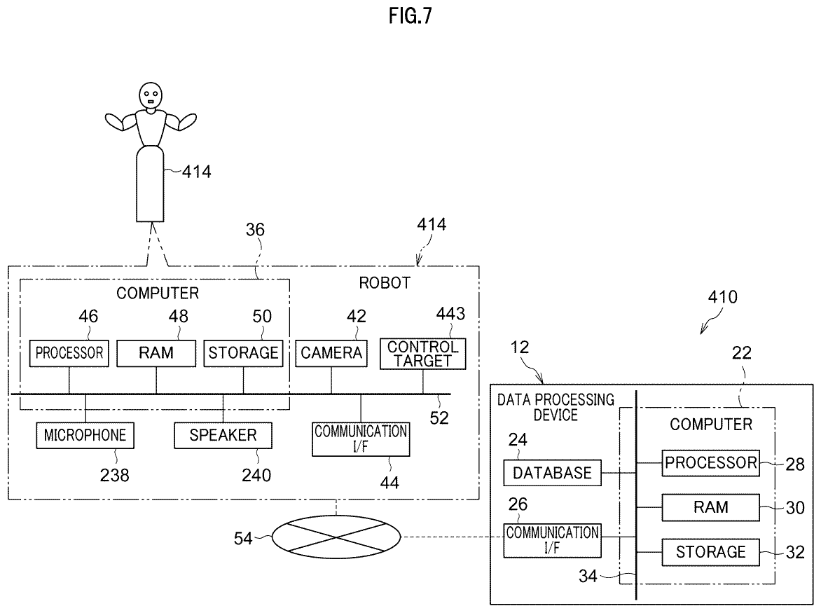

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

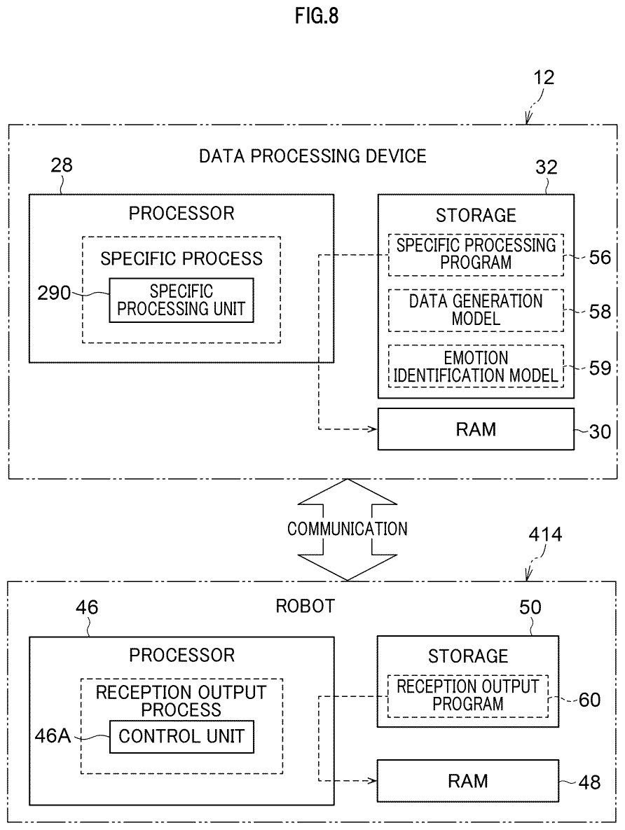

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

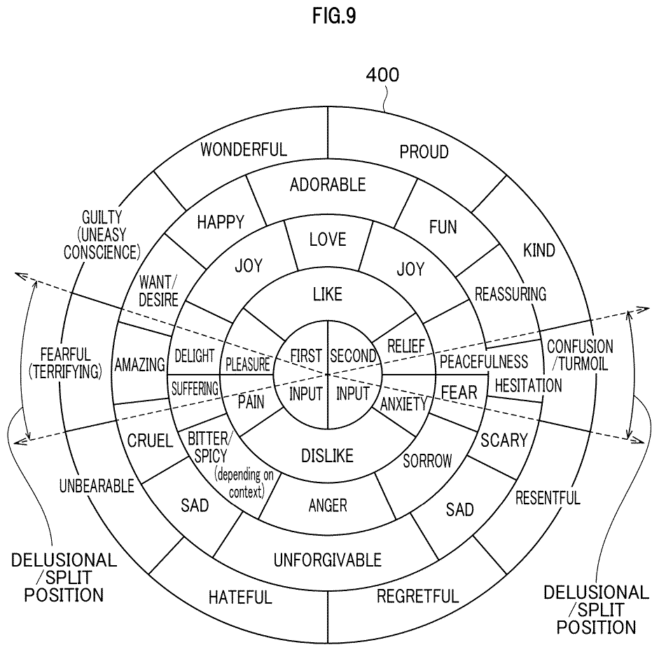

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

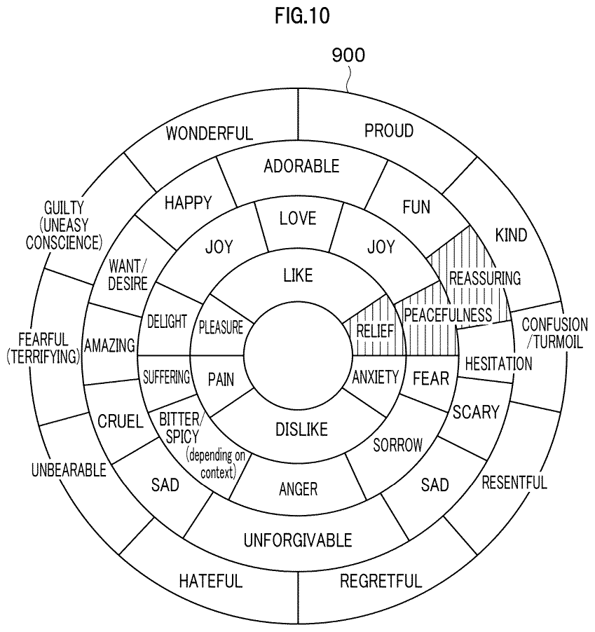

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
receive information acquired from information acquisition devices installed in an observation environment;
perform preprocessing including normalization and removal of abnormal values on the received information using an information processing device to convert the information into a form suitable for analysis;
analyze the preprocessed information to detect behavioral anomalies and biometric variations by means of a learning-enabled information analysis module, and calculate an evaluation index; generate notification information and notify a user using an information presentation device when the evaluation index exceeds a predetermined reference value;
generate and provide countermeasure information to the user together with the notification information;
acquire, through an information input device, feedback information relating to the countermeasures executed by the user, and update the learning-enabled information analysis module based on the feedback information to improve analysis accuracy;
store the information and processing records in an information storage device;
and periodically or as needed transmit and receive information between the information acquisition device and the information processing device via a communication network.

### (Supplementary 2)

The system according to supplementary 1,
wherein the processor is configured to
analyze the preprocessed information in real time to detect behavioral anomalies, and
automatically generate the evaluation index and the notification information.

### (Supplementary 3)

The system according to supplementary 1,
wherein the processor is configured to
aggregate information acquired from a plurality of observation environments and calculate analysis or future predictive information of overall conditions.

### Application Example 1

### (Supplementary 1)

A system comprising a processor,
wherein the processor is configured to
receive information collected from measurement devices installed in multiple animal breeding facilities;
preprocess the received information and convert it into an information format suitable for analysis;
analyze, using the preprocessed information, a generative artificial intelligence model to which a predetermined prompt sentence is input, and obtain numerical results to quantify disease outbreak risk by analyzing abnormal behavior and biological information changes of animals;
generate alert information and provide notification to a manager or other user when the numerical result exceeds a judgment value;
provide preventive measures or responsive actions based on the quantified disease outbreak risk;
acquire result information and emotional state information from a manager or other user, and
update the generative artificial intelligence model and emotion determination engine based on the acquired information; and
send real-time advisory notifications and status reports from the processor to a management terminal.

### (Supplementary 2)

The system according to supplementary 1,
wherein the processor is configured to sequentially analyze the preprocessed information and immediately detect behavioral abnormalities of animals.

### (Supplementary 3)

The system according to supplementary 1,
wherein the processor is configured to aggregate information acquired from a plurality of animal breeding facilities and perform comprehensive situation analysis or future prediction.

### Example 2

### (Supplementary 1)

A system comprising a processor,
wherein the processor is configured to
receive information collected from measuring devices installed in a biological cultivation facility, execute a predetermined algorithm for normalizing and removing abnormal values from the received measurement information and converting the information into a format suitable for analysis,
analyze the pre-processed information using a trained identification model to detect abnormal behaviors and variations in biological indices and quantify the risk of disease occurrence, generate alert information and transmit the alert to a communication terminal when the risk evaluation value exceeds a predetermined threshold,
propose preventive or countermeasure actions based on the alert information and risk evaluation value,
collect feedback and emotional state information transmitted from a human terminal,
update the identification model and emotion estimation model using the collected feedback and emotional state information,
analyze the emotional state of a user and adapt the content of notifications accordingly.

### (Supplementary 2)

The system according to supplementary 1,
wherein the processor is configured to continuously analyze the pre-processed information and detect abnormal behaviors in real time.

### (Supplementary 3)

The system according to supplementary 1,
wherein the processor is configured to integrate information collected from a plurality of biological cultivation facilities and analyze or predict the occurrence of infectious diseases on an aggregate basis.

### Application Example 2

### (Supplementary 1)

A system comprising a processor,
wherein the processor is configured to
receive measurement data collected from information acquisition devices installed in a biological cultivation facility or production facility,
perform preprocessing including noise removal, normalization, and classification on the received measurement data to convert it into a format suitable for information analysis,
analyze the preprocessed measurement data using a generative artificial intelligence model to detect abnormal conditions and quantify an index representing the degree of abnormality,
generate an alert and notify a communication terminal when the index exceeds a preset threshold, present preventive measures or countermeasures based on the index,
receive user response information including action results and emotional state after the notification, and update or retrain the generative artificial intelligence model based on the user response information,
and adjust contents of notifications in accordance with the emotional state of the user.

### (Supplementary 2)

The system according to supplementary 1,
wherein the processor is configured to
analyze the preprocessed measurement data in real time to detect abnormal conditions with minimal time delay.

### (Supplementary 3)

The system according to supplementary 1,
wherein the processor is configured to
integrate measurement data collected from a plurality of biological cultivation or production facilities, and comprehensively analyze and predict overall operational conditions.

What is claimed is:

1. A system comprising a processor,
wherein the processor is configured to:
receive data collected from sensors installed in a livestock farm,
preprocess the received sensor data and convert it into a format suitable for analysis,
analyze, using artificial intelligence, abnormal behavior and body temperature changes based on the preprocessed data and calculate a risk score,
generate and notify an alert when the risk score exceeds a certain threshold,
propose preventive actions and countermeasures based on the risk score, and
collect feedback data from a user and update an artificial intelligence model based on the feedback data.

2. The system according to claim 1, wherein the processor is further configured to analyze the preprocessed data in real-time and detect abnormal behavior.

3. The system according to claim 1, wherein the processor is further configured to aggregate data collected from a plurality of livestock farms and analyze and predict an overall situation.
